(19) United States
(12) Patent Application Publication
(10) Pub. No.: US 2006/0105377 A1
(43) Pub. Date: May 18, 2006

(54) Screening to optimize RNAi

(76) Inventor: Richard M. Eglen, Los Altos, CA (US)

Correspondence Address: PETERS VERNY JONES & SCHMITT, L.L.P., 425 SHERMAN AVENUE, SUITE 230, PALO ALTO, CA, 94306, US

(21) Appl. No.: 11/297,068

(22) Filed: Dec. 7, 2005

Related U.S. Application Data
(63) Continuation-in-part of application No. 10/702,232, filed on Nov. 6, 2003.

(51) Int. Cl. C12Q 1/68

(52) U.S. Cl. 435/6

(57) ABSTRACT

Methods and compositions are provided for screening RNAi molecules for efficiency of modulation, particularly inhibition, of expression of genes. The gene for the target protein is fused to a DNA sequence encoding a small fragment of β-galactosidase. The fragment is competent to complex with a large fragment of β-galactosidase to form an active enzyme. By adding to any of the fusion protein expressed the large fragment and a substrate that produces a detectable product, the amount of detectable product produced is related to the efficiency of modulation of expression by the RNAi. The method finds particular application in high throughput screening.

### CROSS-REFERENCE TO RELATED APPLICATIONS

This application is a continuation-in-part of application Ser. No. 10/702,232, filed Nov. 6, 2003, whose disclosure is incorporated by reference in its entirety.

### REFERENCE TO SEQUENCE LISTING

Applicants assert that the paper copy of the Sequence Listing is identical to the Sequence Listing in computer readable form found on the accompanying computer disk. Applicants incorporate the contents of the sequence listing by reference in its entirety.

### BACKGROUND OF THE INVENTION

1. Technical Field

The field of this invention is screening of RNA compounds for expression inhibition.

2. Background

The elucidation of the human genome and that of other species has greatly accelerated with the interest in proteomics, that is, the study of naturally occurring proteins and their intra- and extracellular interactions and activities. The ability to determine the state or condition of a protein in a cell has far ranging opportunities in understanding the intracellular pathways, the intracellular movement of proteins into different compartments, the regulation of transcription and expression, the regulation of protein content and protein modification, and the like. Not only will this provide greater insight into how a cell operates, but it also allows for the determination of when a cell is aberrant or diseased. In addition, one can determine the effect of changes in the environment of the cell on the cellular function, as evidenced by changes in protein profiles, modification of proteins and transport of proteins.

While it was once suggested that the early biological world was an RNA world and the present world is a DNA world, there has been increasing evidence of RNA having wide ranging activities in regulating biological activity, far greater than associated with expression from mRNA. Of relatively recent date is the discovery of small double stranded RNA (dsRNA) molecules being able to regulate expression by binding to an homologous mRNA and initiating RISC degradation of the mRNA. The RNA interference (RNAi) is a form of post-transcriptional gene silencing, induced by short (19-24 bp) dsRNA sequences that are homologous to mRNA of the silenced gene. Many naturally occurring short RNAs, termed microRNAs (miRNA), have been identified and shown to play active roles in regulating gene expression, especially in development. A family of RNAi molecules has been identified in addition to the miRNAs, such as small hairpin RNAs (shRNA), small interfering RNAs (siRNA), etc. The relative specificity of RNAi and the ability to induce interference by synthetic sequences has greatly expanded interest in RNAi as a research tool, for its potential for use in therapeutics and for controlling the phenotype of cells in a variety of contexts.

Numerous reports of the use of RNAi are present in the patent literature. WO 02/101072 describes methods for modulating the expression of the leucine zipper EF hand transmembrane receptor (LETM-1) and CD43 using siRNA; WO 02/096927 and WO 02/078610 use RNAi to affect the expression of vascular endothelial growth factor receptor (VEGFR) or PAK2, respectively. Other patent references concerned with RNAi include WO 02/085289, WO 03/066650, WO 04/111190, WO 04/063375, WO 04/026227 and WO 05/063980. (All of these references are specifically incorporated by reference.)

Significantly, not all sequences of RNAi are equally effective. There has been substantial effort to devise criteria for designing effective inhibitors. A number of criteria have been established for siRNA, such as differential thermal stability in the ends of the dsRNA molecule, moderate GC content, and the possibility of “position specific” criteria. The criteria for shRNA have been less well characterized. While current design criteria enhance the probability of defining RNAi molecules having enhanced efficacy, there is still the need for improving the selection of criteria and performing functional validation. There is, therefore, a significant need to provide convenient and accurate methods for screening RNA sequences for their ability to modulate, particularly inhibit, expression of proteins in cells.

Relevant Literature

The references cited in the Background are incorporated herein by reference as if fully set forth.

### SUMMARY OF THE INVENTION

Methods, compositions, kits and genetic constructs are provided for intracellularly monitoring a β-galactosidase small fragment containing fusion protein gene as a screen for the inhibition of expression of the fusion protein by double stranded RNA (RNAi). RNAi molecules are screened for their efficiency in modulating expression of a target protein, particularly inhibition. The fusion protein comprises a β-galactosidase enzyme donor oligopeptide fragment (“ED”) fused to a polypeptide sequence representing the target protein, where the activity of the ED in complexing with an enzyme acceptor oligopeptide fragment (“EA”) to form an active β-galactosidase is determined as a measure of the efficiency of inhibition of the RNAi. Double stranded RNA can be formed by transcription from an integrated gene or by addition and transfer across the cellular membrane. The measurement may be intracellular by having a β-galactosidase EA expressed in the cell with substrate present or a lysate may be used.

### BRIEF DESCRIPTION OF THE DRAWINGS

FIG. 1: Constructs and methods used to measure shRNA knockdown activity. (A) Diagram of the co-transfection process used to assess shRNA knockdown activity. An expression plasmid for the gene of interest fused to a reporter sequence is co-transfected with an expression cassette encoding either a specific shRNA (right) or an irrelevant sequence (left). Gene expression changes are measured by comparing the reporter's expression in both sets of transfected cells. (B) Detailed structure of the gene expression constructs. The presence of splice donor (SD) and the splice acceptor (SA) sites, creates an artificial intron that is removed in vivo by the cellular splicing machinery, so that the protein of interest is expressed as a fusion to the reporter sequence. Marked on the intron are SD, 6xHN, Cm^{r}, loxP and prokaryotic promoter elements.

FIG. 2: Initial performance of the ProLabel assay. Transfection-normalized knockdown activity is given for each of 4 shRNAs designed against 17 genes. Averages of triplicate data are shown with standard deviations.

FIG. 3: Analysis of knockdown activity by western blot. STAT1-, STAT6-, and MAPK14-ProLabel (PL) fusions were cotransfected into HEK 293 cells with either a relevant (r) or irrelevant (i) shRNA expression plasmid. After 48 hours, total cell lysates were prepared and subjected to Western analysis. Arrow: STAT1-, STAT6- or MAPK14-specific bands. As a control for loading, the blot was also probed with an antibody against β-actin (actin). The images shown were cropped from the image of the entire gel lane, no other immunoreactive bands were seen. Knockdown percentages, as determined by parallel ProLabel activity assays were: 89%, 86%, and 44% for STAT1, STAT6, and MAPK14, respectively.

FIG. 4: Comparison of ProLabel and qPCR measurements of knockdown activity. Percentage knockdown of 15 different ProLabel fusion proteins, measured at the protein level using the ProLabel assay (grey bars) and at the mRNA level using quantitative RT-PCR (black bars). Data was normalized against secreted luciferase for the ProLabel assay, and against RPLP0 mRNA for the RT-PCR assay. Averages of triplicate data are shown with standard deviations.

FIG. 5: Summary of percentage knockdown obtained from all shRNAs tested for knockdown efficacy. shRNAs were separated into 3 categories according to their effectiveness at knocking down the corresponding gene's expression. The area of each pie segment, represents the proportion of shRNAs meeting the stated level of knockdown. (A) Results from the initial study of 17 genes. (B) Results from the high-throughput analysis of study of 116 genes.

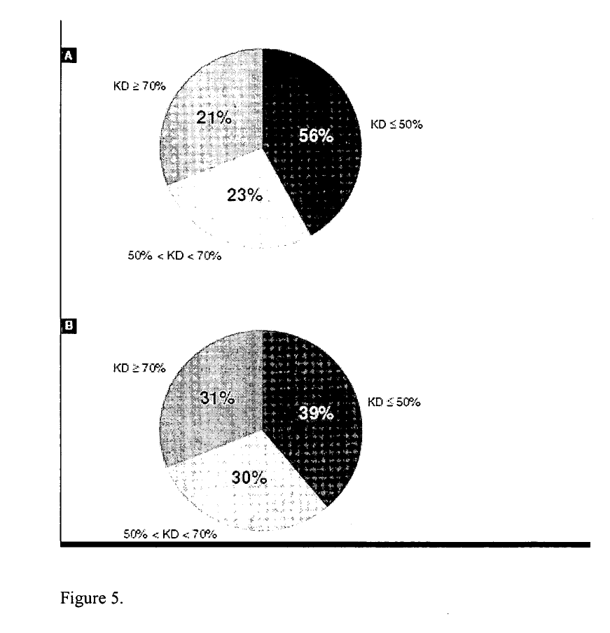

### DESCRIPTION OF THE SPECIFIC EMBODIMENTS

Methods and compositions are provided for modulating, usually inhibiting, expression of target mRNA using ribonucleic acid, particularly RNAi, as dsRNA. RNAi molecules or precursors thereof are screened for efficiency in modulating expression of a target protein(s) in a cell. The method employs a fusion protein that acts as a surrogate or mimic for the mRNA encoding the target protein by fusing at least a major portion of the target protein RNA encoding the target protein with an RNA sequence encoding a small fragment of β-galactosidase (“ED”). The gene encoding the fusion sequence is introduced into a cell transiently or integrated into the genome under conditions for transcription and expression. Also introduced into the cell is a source of the RNAi.

The ED is contacted with the large fragment of β-galactosidase (“EA”), either present in the cell or added in a lysate and also β-galactosidase substrate. The signal resulting from the product of the substrate is related to the efficiency of modulation of expression of the target protein. The method finds particular application is screening a series of RNAi molecules based on a target protein to define the sequence best suited for modulating, usually inhibiting, the expression of the target protein.

A number of features have been reported in the design of RNAi molecules. See, for example, (Meister G and Tuschl T: Mechanisms of gene silencing by double-stranded RNA. Nature 2004; 431:343-349; Murchison E P and Hannon G J: miRNAs on the move: miRNA biogenesis and the RNAi machinery. Curr Opin Cell Biol 2004; 16:223-229; Reynolds A, Leake D, Boese Q, Scaringe S, Marshall W S and Khvorova A: Rational siRNA design for RNA interference. Nat Biotechnol 2004; 22:326-330; Chalk A M, Wahlestedt C and Sonnhammer E L: Improved and automated prediction of effective siRNA. Biochem Biophys Res Commun 2004; 319:264-274; Elbashir S M, Martinez J, Patkaniowska A, Lendeckel W and Tuschl T: Functional anatomy of siRNAs for mediating efficient RNAi in Drosophila melanogaster embryo lysate. Embo J 2001;20:6877-6888; Pancoska P, Moravek Z and Moll U M: Efficient RNA interference depends on global context of the target sequence: quantitative analysis of silencing efficiency using Eulerian graph representation of siRNA. Nucleic Acids Res 2004; 32:1469-1479; Schwarz D S, Hutvagner G, Du T, Xu Z, Aronin N and Zamore P D: Asymmetry in the assembly of the RNAi enzyme complex. Cell 2003; 115:199-208; and Ui-Tei K, Naito Y, Takahashi F, Haraguchi T, Ohki-Hamazaki H, Juni A, Ueda R and Saigo K: Guidelines for the selection of highly effective siRNA sequences for mammalian and chick RNA interference. Nucleic Acids Res 2004; 32:936-948, whose disclosures are incorporated herein by reference.

RNAi molecules are designed in accordance with these concepts as they exist today and may be further improved in the future. See, for example, http://www/rockefeller.edu/labheads/tuschl/sima.html. Open reading frames (“ORF”) are prepared, normally in conjunction with transcriptional regulatory signals, unless the ORF is to be homologously recombined into the target protein of interest. The ORFs include: sequences encoding the RNAi for integration or transient introduction into the host cell; a sequence encoding the fusion protein of the target protein mimetic and ED; and EA for integration or transient introduction, if it is to be provided in the cell. For a lysate analysis, EA may be provided as the protein.

In carrying out the method, after having verified that the constructs are present in the host cell and operating appropriately, the cells are seeded in an appropriate container at a density depending upon the size of the container, generally in the range of about 1×10⁴⁻⁶ cells/well.

For transient transfections, various commercial kits may be employed and the instructions of the supplier followed. Conveniently, the medium for the transfection is DMEM media supplemented with sodium pyruvate, penicillin/streptomycin and 10% fetal bovine serum. Media is replaced at about 6 h after transfection and measurements taken within 48 h. For expression experiments, DNAs are mixed at a mass ratio of about 5:2 (fusion protein:transfection control), while for knockdown experiments mass ratios are about 5:2:1 (fusion protein:transfection control:shRNA). Measurements of expression can be performed using commercial kits (BD Biosciences Clontech) and variations normalized using a secondary reporter, e.g. alkaline phosphatase, luciferase, etc.

The method comprises, after appropriate genetic modification of the host cell, contacting the fusion protein with a β-galactosidase enzyme acceptor in the presence of a detectable substrate, where the β-galactosidase activity is measured. The amount of enzyme product produced is related to the level of expressed ED binding to the EA. The more efficient the RNAi in inhibiting expression, the lower the level of observed enzyme product.

The system employed by the subject invention comprises: (1) preparing the fusion protein gene and expression construct; (2) introducing the expression construct comprising the fusion protein into a selected cell host and providing a transcription construct for production of the RNAi or introducing the RNAi into the host cell; (3) optionally, also introducing an expression construct encoding EA; (4) incubating the transformed cell host under conditions that permit transcription/expression and cell viability; (5) (i) adding a β-galactosidase intracellular substrate or (ii) lysing the cell host and adding EA and a β-galactosidase substrate; and (6) measuring the turnover rate of production of β-galactosidase product as a measure of the efficiency of inhibition of expression

The first component of the subject invention is the fusion protein and its expression construct. The ED may be at either the C-terminus or the N-terminus or internal to the fusion protein. For degradation of the mRNA, it will frequently not matter at what site the ED is situated.

The ED may be inserted into the coding region in a variety of ways. For a cDNA gene, one may select a suitable restriction site for insertion of the sequence, where by using overhangs at the restriction site, the orientation is provided in the correct direction. Alternatively, one may use constructs that have homologous sequences with the target gene and allow for homologous recombination, where the homologous sequences that are adjacent in the target gene are separated by the ED in the construct. By using a plasmid in yeast having the cDNA gene, with or without an appropriate transcriptional and translational regulatory region, one may readily insert the ED construct into the cDNA gene at an appropriate site. Alternatively, one may insert the ED coding region with the appropriate splice sites in an intron or in an exon of the gene encoding the protein of interest. In this way, one can select for a site of introduction at any position in the protein. In some instances, it will be useful to make a number of constructs, where the ED is introduced into an intron and test the resulting proteins for ED activity and availability of the RNA sequence to binding to the RNAi.

Various other conventional ways for inserting encoding sequences into a gene can be employed. For expression constructs and descriptions of other conventional manipulative processes, see, e.g., Sambrook, Fritsch & Maniatis, “Molecular Cloning: A Laboratory Manual,” Second Edition (1989) Cold Spring Harbor Laboratory Press, Cold Spring Harbor, N.Y. (herein “Sambrook et al., 1989”); “DNA Cloning: A Practical Approach,” Volumes I and II (D. N. Glover ed. 1985); “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

The gene encoding the fusion protein will be part of an expression construct. The gene is positioned to be under transcriptional and translational regulatory regions functional in the cellular host. The regulatory region may include an enhancer, which may provide such advantages as limiting the type of cell in which the fusion protein is expressed, requiring specific conditions for expression, naturally being expressed with the protein of interest, and the like. In many instances, the regulatory regions may be the native regulatory regions of the gene encoding the protein of interest, where the fusion protein may replace the native gene, particularly where the fusion protein is functional as the native protein, may be in addition to the native protein, either integrated in the host cell genome or non-integrated, e.g. on an extrachromosomal element.

In those cells in which the native protein is present and expressed, the fusion protein will be competing with the native protein for transcription factors for expression and usually for the RNAi. Therefore, it will be desirable, but not necessary that the endogenous sequences be inactivated from transcription. This can be achieved by knockout of the genes in the host cell, knockout of transcription factors essential for transcription, or the like. The site of the gene in an extrachromosomal element or in the chromosome may vary as to transcription level. Therefore, in many instances, the transcriptional initiation region will be selected to be operative in the cellular host, but may be from a virus or other source that will not significantly compete with the native transcriptional regulatory regions or may be associated with a different gene from the gene for the protein of interest, which gene will not interfere significantly with the transcription of the fusion protein.

It should be understood that the site of integration of the expression construct will affect the efficiency of transcription and, therefore, expression of the fusion protein. One may optimize the efficiency of expression by selecting for cells having a high rate of transcription, one can modify the expression construct by having the expression construct joined to a gene that can be amplified and coamplifies the expression construct, e.g. DHFR in the presence of methotrexate, or one may use homologous recombination to ensure that the site of integration provides for efficient transcription. By inserting an insertion element, such as Cre-Lox at a site of efficient transcription, one can direct the expression construct to the same site. In any event, one will usually compare the β-galactosidase activity from cells in the absence of the RNAi to cells in the presence of RNAi.

There are a large number of commercially available transcriptional regulatory regions that may be used and the particular selection will generally not be crucial to the success of the subject invention. The transcriptional regulatory region may be constitutive or inducible. In the former case, one can have a steady state concentration of the fusion protein in the host, while in the latter case one can provide going from the substantially total absence (there is the possibility of leakage) to an increasing amount of the fusion protein until a steady state is reached. With inducible transcription, one can cycle the cell from a state where the fusion protein is absent to a state where the steady state concentration of the fusion protein is present.

Vectors for introduction of the construct include an attenuated or defective DNA virus, such as but not limited to, herpes simplex virus (HSV), papillomavirus, Epstein Barr virus (EBV), adenovirus, adeno-associated virus (AAV), and the like. Defective viruses, appropriately packaged, which entirely or almost entirely lack viral genes, are preferred. Defective virus is not infective after introduction into a cell. Specific viral vectors include: a defective herpes virus 1 (HSV1) vector (Kaplitt et al., 1991, Molec. Cell. Neurosci. 2:320-330); an attenuated adenovirus vector, such as the vector described by Stratford-Perricaudet et al. (1992, J. Clin. Invest. 90:626-630 a defective adeno-associated virus vector (Samulski et al., 1987, J. Virol. 61:3096-3101; Samulski et al., 1989, J. Virol. 63:3822-3828).

The vector may be introduced in vitro by lipofection. For the past decade, there has been increasing use of liposomes for encapsulation and transfection of nucleic acids in vitro. (Felgner, et. al., 1987, Proc. Natl. Acad. Sci. U.S.A. 84:7413-7417; see Mackey, et al., 1988, Proc. Natl. Acad. Sci. U.S.A. 85:8027-8031). The use of cationic lipids may promote encapsulation of negatively charged nucleic acids, and also promote fusion with negatively charged cell membranes (Felgner and Ringold, 1989, Science 337:387-388). Targeted peptides or non-peptide molecules can be coupled to liposomes chemically.

It is also possible to introduce the vector in vitro as a naked DNA plasmid, using calcium phosphate precipitation or other known agent. Alternatively, the vector containing the gene encoding the fusion protein can be introduced via a DNA vector transporter (see, e.g., Wu et al., 1992, J. Biol. Chem. 267:963-967; Wu and Wu, 1988, J. Biol. Chem. 263:14621-14624; Hartmut et al., Canadian Patent Application No. 2,012,311, filed Mar. 15, 1990). The same manner in which the fusion protein construct is introduced can be used for the gene encoding the RNAi. The same vector can be used for both constructs.

Vectors are introduced into the desired host cells in vitro by methods known in the art, e.g., transfection, electroporation, microinjection, transduction, cell fusion, DEAE dextran, calcium phosphate precipitation, lipofection (lysosome fusion), use of a gene gun, using a viral vector, with a DNA vector transporter, and the like.

Expression vectors containing the fusion protein gene inserts can be identified by four general approaches: (a) PCR amplification of the desired plasmid DNA or specific mRNA, (b) nucleic acid hybridization, (c) presence or absence of “marker” gene functions, and (d) expression of inserted sequences. In the first approach, the nucleic acids can be amplified by PCR with incorporation of radionucleotides or stained with ethidium bromide to provide for detection of the amplified product. In the second approach, the presence of the fusion protein gene inserted in an expression vector can be detected by nucleic acid hybridization using probes comprising sequences that are homologous to the fusion protein gene. In the third approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of foreign genes in the vector. In the fourth approach, recombinant expression vectors can be identified by assaying for the activity of the fusion protein gene product expressed by the recombinant expression vector. Similarly, the presence of the construct for the RNAi in the host cell may be verified.

One may use promoters that are active for a short time, such as viral promoters for early genes, for example, the human cytomegalovirus (CMV) immediate early promoter. Other viral promoters include but are not limited to strong promoters, such as cytomegaloviral promoters (CMV), SR.alpha. (Takebe et al., Mol. Cell. Biol. 8:466 (1988), SV40 promoters, respiratory syncytial viral promoters (RSV), thymine kinase (TK), beta-globin, etc. Alternatively, an inducible promoter can be used.

A large number of promoters have found use in various situations, for various purposes and for various hosts. Many promoters are commercially available today. Expression of the fusion protein may be controlled by any promoter/enhancer element known in the art, but these regulatory elements must be functional in the host or host cell selected for expression. Promoters which may be used to control fusion gene expression include, but are not limited to, the SV40 early promoter region (Benoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42); and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58), alpha 1-antitrypsin gene control region which is active in the liver (Kelsey et al., 1987, Genes and Devel. 1: 161-171), beta-globin gene control region which is active in myeloid cells (Mogram et al., 1985, Nature 315:338-340; Kollias et al., 1986, Cell 46:89-94), myelin basic protein gene control region which is active in oligodendrocyte cells in the brain (Readhead et al., 1987; Cell 48:703-712), myosin light chain-2 gene control region which is active in skeletal muscle (Sani, 1985, Nature 314:283-286), prostate specific antigen control region, which is active in prostate cells (U.S. Pat. Nos. 6,197,293 and 6,136,792), and gonadotropic releasing hormone gene control region which is active in the hypothalamus (Mason et al., 1986, Science 234:1372-1378).

Alternatively, expression of the fusion protein gene can be under control of an inducible promoter, such as metallothionine promoter, which is induced by exposure to heavy metals. For control of the gene transfected into certain brain cells, a glucocorticoid inducible promoter can be used. Alternatively, an estrogen inducible promoter, which would be active with cells from the hypothalamus and other areas responsive to estrogen.

Similar considerations are applicable for the RNAi ORF. By using promoters that provide for comparable or greater transcription of the RNAi than the fused protein, one can ensure that there is a sufficient amount of effective inhibitory RNAi to bind to the fusion protein mRNA to inhibit transcription at a detectable level.

Vectors containing DNA encoding the following proteins, for example, have been deposited with the American Type Culture Collection (ATCC) of Rockville, Md.: Factor VIII (pSP64-VIII, ATCC No. 39812); a Factor VIII analog, “LA”, lacking 581 amino acids (pDGR-2, ATCC No. 53100); t-PA and analogs thereof (see co-pending U.S. application Ser. No. 882,051); VWF (pMT2-VWF, ATCC No. 67122); EPO (pRK1-4, ATCC No. 39940; pdBPVMMTneo 342-12 (BPV-type vector) ATCC No. 37224); and GM-CSF (pCSF-1, ATCC No. 39754).

The vector will include the fusion gene under the transcriptional and translational control of a promoter, usually a promoter/enhancer region, optionally a replication initiation region to be replication competent, a marker for selection, as described above, and may include additional features, such as restriction sites, PCR initiation sites, an expression construct providing constitutive or inducible expression of EA, or the like. As described above, there are numerous vectors available providing for numerous different approaches for the expression of the fusion protein in a host.

The host cells will be selected to provide the necessary transcription factors for expression of the fusion protein and the other components for the purposes of the determination. In most cases, established cell lines will be used, since the cell lines can provide the desired environment and allow for direct comparisons between studies, which comparisons may not be available where using primary cell lines from patients. Established cell lines, including transformed cell lines, are suitable as hosts. Normal diploid cells, cell strains derived from in vitro culture of primary tissue, as well as primary explants (including relatively undifferentiated cells such as hematopoietic stem cells) are also suitable. Embryonic cells may find use, as well as stem cells, e.g. hematopoietic stem cells, neuronal stem cells, muscle stem cells, etc. Candidate cells need not be genotypically deficient in a selection gene so long as the selection gene is dominantly acting. The host cells preferably will be established mammalian cell lines. For stable integration of vector DNA into chromosomal DNA, and for subsequent amplification of the integrated vector DNA, both by conventional methods, CHO (Chinese Hamster Ovary) cells are convenient. Alternatively, vector DNA may include all or part of the bovine papilloma virus genome (Lusky et al., 1984, Cell 36:391-401) and be carried in cell lines such as C127 mouse cells as a stable episomal element. Other usable mammalian cell lines include HeLa, COS-1 monkey cells, melanoma cell lines such as Bowes cells, mouse L-929 cells, mouse mammary tumor cells, 3T3 lines derived from Swiss, Balb-c or NIH mice, BHK or HAK hamster cell lines and the like.

Cell lines may be modified by knocking out specific genes, introducing specific genes, e.g. the EA coding gene, enhancing or diminishing the expression of a protein or the like. The modification may be transient, as in the case of introduction of antisense DNA or dsRNA, including RNAi, such as siRNA, or may be permanent, by deleting a gene, introducing a gene encoding the antisense mRNA of the target protein, adding a dominant recessive gene, or the like. These procedures are well established as evidenced by the scientific and patent literature. See, for example, for antisense: Zhang, et al., 2002 J Gene Med 4. 183-94; Shi, et al., 2001 Cancer Biother Radiopharm 16,421-9; Allen and Renzi 20021 Antisense Nucleic Acid Drug Dev 11, 289-300; WO 00/61602; WO99/61462; and WO92/00990; for dsRNA, Heitmeier, et al.1999 J Biol Chem 274, 12531-6, US2002/0114784 and WO 01/77350; and a special case of dsRNA, namely iRNA: Agami, 2002 Curr Opin Chem Biol 6, 829-34; Minski, et al., J Biol Chem 277, 49453-8; Malhotra, et al., 2002 Mol Microbiol 45, 1245-54; Sui, et al., 2002 PNAS USA 99, 5515-20; and Yang, et al., 2000 Curr Biol 10, 1191-2000. Methods for introducing the RNA transiently are well known as exemplified by the references cited above. For permanent integration, the methods described in the above references can be employed.

The ED is extensively described in the patent literature. U.S. Pat. Nos. 4,378,428; 4,708,929; 5,037,735; 5,106,950; 5,362,625; 5,464,747; 5,604,091; 5,643,734;and PCT application nos. WO96/19732; and WO98/06648 describe assays using complementation of enzyme fragments. The ED will generally be of at least about 35 amino acids, usually at least about 37 amino acids, frequently at least about 40 amino acids, and usually not exceed 100 amino acids, more usually not exceed 75 amino acids. The upper limit is defined by the effect of the size of the ED on the performance and purpose of the determination, the effect on the complementation with the EA, the inconvenience of a larger construct, and the like. The minimum size that can be used must provide a signal that is modulated by the cellular events and that can be determined with reasonable sensitivity.

Of the protein categories of interest, transcription factors, inhibitors, regulatory factors, enzymes, membrane proteins, structural proteins, and proteins complexing with any of these proteins, are of interest. Specific proteins include enzymes, such as the hydrolases exemplified by amide cleaving peptidases, such as caspases, thrombin, plasminogen, tissue plasminogen activator, cathepsins, dipeptidyl peptidases, prostate specific antigen, elastase, collagenase, exopeptidases, endopeptidases, aminopeptidase, metalloproteinases, including both the serine/threonine proteases and the tyrosine proteases; hydrolases such as acetylcholinesterase, saccharidases, lipases, acylases, ATP cyclohydrolase, cerebrosidases, ATPase, sphingomyelinases, phosphatases, phosphodiesterases, nucleases, both endo- and exonucleases; oxidoreductases, such as the cytochrome proteins, the dehydrogenases, such as NAD dependent dehydrogenases, xanthine dehyrogenase, dihydroorotate dehydrogenase, aldehyde and alcohol dehydrogenase, aromatase; the reductases, such as aldose reductase, HMG-CoA reductase, trypanothione reductase, etc., and other oxidoreductases, such as peroxidases, such as myeloperoxidase, glutathione peroxidase, etc., oxidases, such as monoamine oxidase, myeloperoxidases, and other enzymes within the class, such as NO synthase, thioredoxin reductase, dopamine β-hydroxylase, superoxide dismutase, nox-1 oxygenase, etc.; and other enzymes of other classes, such as the transaminase, GABA transaminase, the synthases, β-ketoacyl carrier protein synthase, thymidylate synthase, synthatases, such as the amino acid tRNA synthatase, transferases, such as enol-pyruvyl transferase, glycinamide ribonucleotide transformylase, COX-1 and -2, adenosine deaminase.

A number of substrates for β-galactosidase are known, where the product is fluorescent. The common substrates are β-D-galactopyranosyl phenols, such as fluorescein, mono- and di-susbtituted, o-nitrophenyl-β-D-galactoside, β-methylumbelliferyl-β-D-galactoside, X-gal, resorufin-β-D-galactoside, commercially available oxetanes, e.g. Galacto-Light Plus® kits (chemiluminescence) and chlorophenol red. The di-β-D-galactopyranosylfluorescein, and chlorophenol red-β-D-galactopyranoside, or analogous substrates, particularly where the product is inhibited from leaking from the cell, may be used as intracellular markers.

During the determination, the cells are maintained in a viable state, where the cells may be dividing or not dividing. The viable state may be referred to growing. The determination may be made with intact cells or a cellular lysate.

With intact cells, the cells are maintained in the culture, during which time the fusion protein and EA are expressed intracellularly, either transiently, constitutively or inducibly. Also, the substrate will be maintained, usually in the medium at a concentration where the substrate in the cell is at a concentration that will permit detection of changes in the activity of the fusion protein relevant to the assay. In some instances, one can inject the substrate into the cell using any conventional technique or provide for permeabilization of the cell, followed by washing and curing the membrane, so as to lock the substrate intracellularly. The cells can be analyzed by FACS, electrophoretically, fluorimetrically, etc.

For convenience, kits can be provided that may include all or some of the major components of the assays. For example, a kit may include an expression construct, by itself or as part of a vector, e.g. plasmid, virus, usually attenuated, where the expression construct may include a marker, an ORF encoding a RNAi for integration, a replication initiation site, and the like. In addition to the expression construct, the kit may include EA, substrate for β-galactosidase, one or more cell lines or primary cells, a graph of response in relation to the amount of ED present, buffer, etc. In some instances cells may be engineered to provide a desired environment, such as high levels of expression of a protein related to the target protein, such as surface membrane receptors, GPCRs, nuclear receptors, e.g. steroid receptors, transcription factors, etc. or may have been mutated, so as to have reduced levels of expression affecting the expression of the fusion protein and one is interested in enhancing the level of expression.

The system can be used with a data accumulation and storage capability, where the data derived from the system is collected, analyzed and compared to other determinations. In this way, data can be accumulated of the effect of various sequences on the efficiency of RNAi molecules, so that one can measure the characteristics to be considered in designing RNAi molecules. By having a database of known responses to changes in sequence of the RNAi, new RNAi molecules may be designed with greater success in efficient inhibition.

The following examples are offered by way of illustration and not by way of limitation.

### EXPERIMENTAL

The following work was performed at BD Clontech Laboratories following procedures set forth in the subject application.

Methods

Fusion-protein Expression Vector Construction

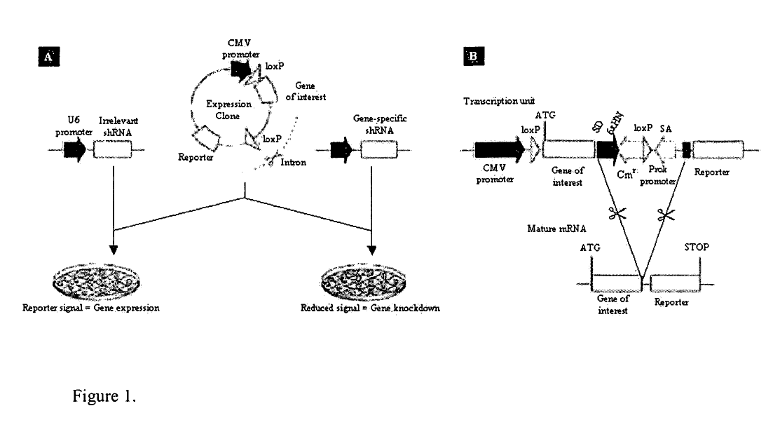

Full-length, sequence verified open reading frames (ORFs) for all 133 genes used in this study are part of a collection of more than 1600 human ORFs (Creator Clone Collection) available from Open Biosystems (Huntsville, Ala.). All sequence data on these ORFs are available in GenBank. Each ORF is cloned into a specialized cloning vector (pDNR-Dual, Clontech Laboratories, Mountain View, Calif.) that enables rapid transfer of the ORFs to appropriately designed expression vectors using Cre/LoxP-based recombination. To enable fusions to peptide reporters at the C-terminus of the protein, each ORF was cloned into pDNR-DUAL such that the natural stop codon was removed and replaced with codon for leucine. To express the proteins of interest as fusions to a reporter tag, the ORFs were transferred by Cre/LoxP-based recombination from the pDNR-Dual backbone into an expression vector that placed the reporter tag in-frame and C-terminal to the gene of interest (FIGS. 1A & 1B). A synthetic intron between the end of the gene and the start of the tag is removed in vivo by cellular splicing machinery, generating an expressed mRNA encoding the protein of interest fused at its C-terminus to the reporter. The reporter tag used was a modified alpha fragment of β-galactosidase, ProLabel (DiscoveRx, Fremont, Calif.). To ensure that expression of the ProLabel tag was strictly dependent on expression of the protein of interest, it had been cloned into the expression vector (pLPS-3′PL; Clontech Laboratories) lacking its own initiation codon. Following recombination and transformation, colonies were screened for recombinants by triplex PCR using the following primers:
(CMV1:5′ACTCCGCCCCATTGACGCAA, (SEQ ID NO:1) PCP2: 5′
TCCGCTCATGAGACAATAACC, (SEQ ID NO:2) PCP3:
5′ CACCTTGTCGCCTTGCGTAT, (SEQ ID NO:3) SacB forward: 5′
GACGATTGACGGCATTACGT, (SEQ ID NO:4) SacB reverse: 5′
TGCCTTTGATGTTCAGCAGG) (SEQ ID NO:5). Expected PCR products are 269 bp for non-recombinant pDNR-Dual (SacB forward/SacB reverse); 177 bp for non-recombinant acceptor vectors (CMV1 and PCP2); and 661 bp for the recombinants (PCP2 and PCP3).
   Design of shRNAs

Four shRNA-encoding sequences were selected for each gene tested. Based on previous studies, which have shown efficient knockdown using shRNAs with 19bp stems, (Hannon G J and Rossi J J: Unlocking the potential of the human genome with RNA interference. Nature 2004; 431:371-378; Brummelkamp T R, Bernards R and Agami R: A system for stable expression of short interfering RNAs in mammalian cells. Science 2002; 296:550-553; Miyagishi M and Taira K: U6promoter-driven siRNAs with four uridine 3′ overhangs efficiently suppress targeted gene expression in mammalian cells. Nat Biotechnol 2002; 20:497-500; and Paul C P, Good P D, Winer I and Engelke D R: Effective expression of small interfering RNA in human cells. Nat Biotechnol 2002; 20:505-508.), we chose to use a stem length of 19 bases for our shRNA designs. Specificity of shRNA 19-mer sense oligonucleotides was confirmed by sequence similarity search against the NCBI collection of human genes (RefSeq release November 2003). Both the sense and antisense orientations of each sequence were searched to reduce the possibility of off-target effects caused by homology of either strand to occult target mRNAs. All selected oligonucleotide sequences were free of genomic repeats and had no similarity longer than 13 bases to any secondary target mRNA in the collection in either the sense or antisense strand. In addition, an effort was made to allocate each of the four shRNA sequences evenly throughout the mRNA length, as far from each other as possible within the coding region of the target mRNA. Initially, shRNA oligonucleotides were designed using the basic rules described by Tuschl and collaborators (see http://www.rockefeller.edu/labheads/tuschl/sima.html) using an on-line design tool (http://bioinfo.clontech.com/rnaidesigner/). Based on our initial results and those published by others, (Reynolds A, Leake D, Boese Q, Scaringe S, Marshall W S and Khvorova A: Rational siRNA design for RNA interference. Nat Biotechnol 2004; 22:326-330; Schwarz D S, Hutvagner G, Du T, Xu Z, Aronin N and Zamore P D: Asymmetry in the assembly of the RNAi enzyme complex. Cell 2003; 115:199-208; and Ui-Tei K, Naito Y, Takahashi F, Haraguchi T, Ohki-Hamazaki H, Juni A, Ueda R and Saigo K: Guidelines for the selection of highly effective siRNA sequences for mammalian and chick RNA interference. Nucleic Acids Res 2004; 32:936-948.), a multi-step selection procedure was selected implementing a set of sequence constraints on all gene-specific 19-mers identified. These constraints were as follows: 1) No stretch of the same base longer than 4 was permitted; 2) All 4 bases had to appear at least once, but not more than ten times, in any oligonucleotide. In addition, a low complexity filter was applied to eliminate sequences of alternating nucleotides (e.g., ACACACAC (SEQ ID NO:6) or AACCAACC (SEQ ID NO:7)). Filtered sequences were then ranked according to GC-content (min=30%, opt=40%, max=52%), Tm of sense-antisense RNA duplex (min=50C, opt=55C, max=60C), and Tm of any internal RNA hairpins (max=50C). Tm values were calculated using the nearest-neighbor model according to Mathews et al. (Mathews D H, Sabina J, Zuker M and Turner D H: Expanded sequence dependence of thermodynamic parameters improves prediction of RNA secondary structure. J Mol Biol 1999; 288:911-940.)

It has been shown that siRNAs having a lower thermal stability at the 3′ end of the sense-strand with respect to the 5′ end promote incorporation of the desired anti-sense-strand into the RISC complex, and thus show improved knockdown efficacy compared to sequences lacking such thermal asymmetry. As a simple approach to preferentially selecting sequences for shRNAs with similar asymmetry in their thermal stability, sequences with an A or U in positions 17 to 19 of the sense oligonucleotide were preferentially selected over others. Overall, the ranking system did not strictly enforce selection of oligonucleotides within the optimal parameters, but rather provided a basis for compromise when no “ideal” sequence could be found.

For each 19 base pair sequence chosen, a pair of complementary DNA oligonucleotides of 65 to 67 bases in length encoding the required shRNA sequence was synthesized and PAGE purified (Sigma-Genosys, Woodlands, Tex.; or Integrated DNA Technology, Coralville, Iowa). Each oligonucleotide pair included the following elements: a Bam HI overhang on the 5′ end of the duplex; the 19 nucleotides of shRNA sense strand; a loop sequence (top strand: 5′ TTCAAGAGA); the 19 nucleotides of the shRNA antisense strand; a Poll III termination site of 6 consecutive thymidine residues; an Nhe I or Mlu I site to verify cloned inserts; and an Eco RI overhang on the 3′ end of the duplex. It has been shown that PolIII transcription initiates only from purines (Lobo S M, Ifill S and Hernandez N: cis-acting elements required for RNA polymerase II and III transcription in the human U2 and U6snRNA promoters. Nucleic Acids Res 1990; 18:2891-2899.). Thus, if the designed 19-nucleotide shRNA sequence did not start with a guanine or adenine, an extra guanine residue was added to the 5′ end of the shRNA sense strand, and this 20-nucleotide sense-strand was then used in place of the 19 base sequence as a basis for oligo design. Retrospectively, we examined the effect of this additional guanine, by comparing the knockdown activity of 3 shRNAs with or without the initial guanine. In all three cases, removal of the guanine resulted in a dramatic decrease in shRNA efficacy, suggesting that, as reported, POLIII-based transcription shows a strong preference to start at a purine.

Generation of shRNA Plasmids and Expression Cassettes

Annealed oligonucleotides encoding shRNAs were either cloned by standard ligation methods into an shRNA expression vector (pSIREN-DNR; Clontech Laboratories, Mountain View, Calif.) or used to generate linear shRNA expression cassettes (SECs). For SEC production, annealed oligonucleotides encoding shRNAs were ligated into pSIREN-DNR, as described in the Clone & Confirm Kit user manual (Clontech Laboratories, Mountain View, Calif.). Then, 1 μL of the ligation was amplified by PCR using the following vector-specific primers: fwd=5′-CCTGCGTTATCCCCTGATTCTGTG′ (SEQ ID NO:8); rev=5′-CAGGGCGGGGCGTAATTTGATATC (SEQ ID NO:9). Annealing was done at 60° C. for 40 seconds and extensions at 72° C. for 1 minute, for a total of 30 cycles. For high-throughput cloning, PCR reactions were performed in parallel in a 96-well plate using a lyophilized Taq polymerase enzyme formulation (SPRINT Advantage 96-well plates; Clontech Laboratories).

For each shRNA expression vector cloned, a total of three colonies were picked and screened by restriction digest for the presence of an insert using either Nhe I or Mlu I. One insert-containing clone for each shRNA was then additionally verified by sequencing. SECs were screened by size for presence of the insert using a 2% agarose gel. For transfections, plasmids were purified using NucleoSpin® plasmid purification kit (Clontech Laboratories), whereas SECs were purified following PCR using the NucleoSpin® Extract Kit (Clontech Laboratories). For both plasmids and SECs, expression of the shRNA was driven by the human U6 promoter (Accession: M14486) (Kunkel G R, Maser R L, Calvet J P and Pederson T: U6 small nuclear RNA is transcribed by RNA polymerase III. Proc Natl Acad Sci USA 1986; 83:8575-8579; and Kunkel G R and Pederson T: Transcription of a human U6 small nuclear RNA gene in vivo withstands deletion of intragenic sequences but not of an upstream TA TA TA box. Nucleic Acids Res 1989; 17:7371-7379).

Analysis of knockdown By Transient Cotransfection

Transient transfections to measure either protein expression or knockdown were done using HEK293 cells seeded 24-48 hours prior to transfection in either 12-well or 96-well plates (FIG. 1). Cells were seeded at a density of either 2-2.5×105 cells/well (12-well plates) or 2-3×104 cells/well (96-well plates). For assays done in 12-well plates, transfections were done using the CalPhos Mammalian Transfection Kit (Clonetech Laboratories, Mountain View, Calif.), according to the protocol in the user manual. In 12 well plates, 125 μL transfection mix containing 800 ng of total DNA was added to each well containing 1 mL DMEM media supplemented with 5 mM sodium pyruvate, penicillin/streptomycin, and 10% fetal bovine serum. In 96-well plates, transfections were done using Lipofectamine 2000™ (Invitrogen, Carlsbad, Calif.), following the manufacturer's instructions, with a total of 150 ng DNA per well. In all cases, media was replaced 6 hours after transfection, and expression measurements were performed after 48 hours. Except where noted, all transfections were done in triplicate, and data are presented as the average of triplicate transfections±standard deviation.

For expression experiments, DNAs were mixed at a mass ratio of 5:2 (fusion protein:transfection control). For knockdown experiments, DNAs were mixed at a mass ratio of 5:2:1 (fusion protein:transfection control:shRNA). Previous experiments (data not shown) had demonstrated that efficacious shRNAs caused knockdown in transient-transfection experiments even when the vector expressing them was transfected at a low mass ratio in comparison to the target gene expression construct. Thus, to bias experiments in favor of identifying highly effective shRNAs, transfections were done using an excess of the target construct DNA relative to the shRNA expression vector.

Reporter Expression Measurements

To quantify expression of the fusion proteins, β-galactosidase activity was measured using the ProLabel Chemiluminescent Detection Kit (Clontech Laboratories, Mountain View, Calif.), following the manufacturer's instructions. For all experiments, variations in transfection efficiency were normalized by co-transfecting the experimental plasmids with a secondary reporter. Mostly, pCMV-SEAP (Clontech Laboratories) was used as the secondary reporter. In this case, culture medium was collected from the cells 48 hours post-transfection and assayed for secreted alkaline phosphatase (SEAP) activity using the Great EscAPe SEAP Chemiluminescent Detection Kit (Clontech Laboratories). In some instances, a cellular luciferase reporter was used (pCMV-Luc), and luciferase activity was measured in the cell lysates using firefly luciferin (Promega, Madison, Wis.). Finally, in some experiments, a secreted luciferase reporter was used with coelenterazine (Coelenterate luciferin, Promega) as substrate in a reaction as previously described. (Markova S V, Golz S, Frank L A, Kalthof B and Vysotski E S: Cloning and expression of cDNA for a luciferase from the marine copepod Metridia longa. A novel secreted bioluminescent reporter enzyme. J Biol Chem 2004; 279:3212-3217). All chemiluminescent signals were quantified on a Monolight 3096 plate luminometer (BD Biosciences Pharmingen, La Jolla, Calif.).

Measurement of Protein Knockdown

Percentage knockdown induced at the protein level by each shRNA was calculated by taking the normalized ProLabel activity measured in cells transfected with the shRNA (SEC or plasmid) specific for the gene-of-interest and comparing it to the ProLabel activity measured in cells transfected with an ‘irrelevant’ shRNA directed against luciferase. The sequence of the sense-strand of this ‘irrelevant’ shRNA is: 5′ GTGCGTTGCTAGTACCAAC (SEQ ID NO:10).

Statistical Analysis

Statistical analysis of the effects of various design criteria on knockdown activity was done using the rank-sum test. Student's t-test was used to assess whether the effectiveness of each shRNA could be considered independent and to test the significance of the difference in thermal asymmetry between effective and ineffective shRNAs.

Real-time, Quantitative PCR Assays

In some cases, knockdown was measured by real-time quantitative PCR in addition to the ProLabel reporter assay. To do this, cells were removed from the culture-plate with Dulbecco's PBS supplemented with 1 mM EDTA and split into two equal portions. One portion was then lysed and assayed for ProLabel reporter activity, as described above.

Total RNA was extracted from the second portion with the NucleoSpin II RNA extraction kit (Clontech Laboratories, Mountain View, Calif.). From this RNA, first-strand cDNA was generated by random-primed reverse transcription using PowerScript reverse-transcriptase (Clontech Laboratories). Expression of mRNA was then determined by real-time quantitative PCR using a technology based on the catalytic activity of a DNAzyme (Breaker R R and Joyce G F: A DNA enzyme with Mg(2+)-dependent RNA phosphoesterase activity. Chem Biol 1995; 2:655-660; Joyce G F: Directed evolution of nucleic acid enzymes. Annu Rev Biochem 2004; 73:791-836). (QZyme technology, Clontech Laboratories). Primer pairs were designed around the shRNA target-site in the gene of interest, to ensure that the shRNA-induced mRNA cleavage was directly measured. All assays were run in duplex mode on an ABI 7700, using primers to Ribosomal Protein, large, P0 (RPLP0) to normalize gene expression. Full details of the method can be found at the following URL:

http://www.bdbiosciences.com/clontech/techinfo/manuals/PDF/PT3780-1.pdf.

Knockdown activity was determined by comparing normalized mRNA expression in the presence of the shRNA of interest to that in the presence of an irrelevant shRNA.

Western Blot Analysis

48 hours post-transfection, cells were lysed, and the lysates used either for measurement of ProLabel activity or western blot analysis using specific antibodies for STAT1, STAT6, MAPK14 and β-actin (BD Biosciences Pharmingen, La Jolla, Calif.). Briefly, about 15 μg per lane of each lysate were separated on 4-20% gradient 10-well minigels (Invitrogen, Carlsbad, Calif.), transferred to PVDF membranes, and probed with each antibody at the recommended optimal concentration using a standard western blot protocol.

### RESULTS

Use of the ProLabel Tag to Screen for Knockdown

The subject method is focused on a small, 55 amino acid, N-terminal fragment of β-galactosidase (ProLabel, DiscoveRx, Fremont, Calif.) that can be used to reconstitute the enzymatic activity of an inactive C-terminal (□) fragment (enzyme acceptor; EA). This restored activity is readily quantified using standard chemiluminescent β-galactosidase substrates. Three features of this assay make it especially useful for high-throughput analysis. First, it is a homogenous assay. Second, all genes can be analyzed under the same conditions. Finally, the signal generated can be read in any standard plate-based luminometer.

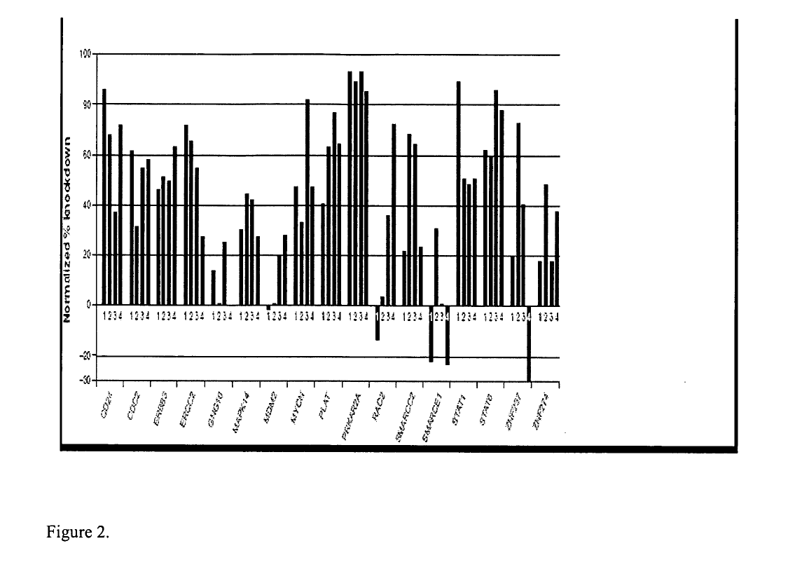

The effectiveness of the ProLabel tag as a measure of protein knockdown was shown by generating ProLabel-tagged expression constructs for 17 genes and confirmed expression by transient transfection of HEK293 cells (data not shown). Using rules described by Tuschl et al. four shRNAs were designed against each of the genes (total of 68 shRNAs) and cloned into a pre-linearized shRNA expression vector (pSIREN-DNR, Clontech Laboratories, Mountain View, Calif.). Clones were verified by restriction digestion and sequencing. Each cloned shRNA was then screened for efficacy by co-transfection with the respective ProLabel fusion construct (see Methods and FIG. 1). As shown in FIG. 2, some shRNAs were highly effective (knockdown activity >70%), whereas others had less effect. In the case of some genes, e.g., SMARCE1, lack of knockdown may be attributed to poor expression of the ProLabel construct, making accurate measurement of knockdown difficult. Another possible explanation for the failure of some shRNAs to induce knockdown is that the 19 bp stems of the shRNAs are too short for effective processing into siRNAs. To address this concern, we sought to test the effect of increasing stem length on the efficacy of poorly functional shRNAs. Stem lengths of 21, 23 and 27 bases were tested for several different shRNAs against seven of the genes. However, no consistent improvement in knockdown activity was observed. This result is consistent with the findings of other researchers (Brummelkamp T R, Bemards R and Agami R: A system for stable expression of short interfering RNAs in mammalian cells. Science 2002; 296:550-553; Miyagishi M and Taira K: U6promoter-driven siRNAs with four uridine 3 ′ overhangs efficiently suppress targeted gene expression in mammalian cells. Nat Biotechnol 2002; 20:497-500; Paul C P, Good P D, Winer I and Engelke D R: Effective expression of small interfering RNA in human cells. Nat Biotechnol 2002; 20:505-508); though recently two publications have presented evidence suggesting that longer stems may be beneficial (Kim D H, Behlke M A, Rose S D, Chang M S, Choi S and Rossi J J: Synthetic dsRNA Dicer substrates enhance RNAi potency and efficacy. Nat Biotechnol 2005; 23:222-226; Kim D H, Behlke M A, Rose S D, Chang M S, Choi S and Rossi J J: Synthetic dsRNA Dicer substrates enhance RNAi potency and efficacy. Nat Biotechnol 2005; 23:222-226; Siolas D, Lerner C, Burchard J, Ge W, Linsley P S, Paddison P J, Hannon G J and Cleary M A: Synthetic shRNAs as potent RNAi triggers. Nat Biotechnol 2005; 23:227-231; Siolas D, Lerner C, Burchard J, Ge W, Linsley P S, Paddison P J, Hannon G J and Cleary M A: Synthetic shRNAs as potent RNAi triggers. Nat Biotechnol 2005; 23:227-23 1. Nonetheless, Brummelkamp et al. supra, have shown that shRNAs with 19 bp stems are rapidly converted in the cell to siRNA-like molecules of approximately 19 bases in length. Based on these results, we conclude that 19 base stems are adequate and that lack of activity is not simply due to inefficient processing of the shRNAs.

For reasons that remain unclear, some shRNAs appeared to induce expression of the gene of interest (e.g., certain shRNAs against ZNF237 and RAC2). Overall, 30 of the shRNAs (44%) reduced gene expression by at least 50%. Of these, 14 (21%) induced knockdown by at least 70%. In total, 9 genes were identified for which at least one shRNA gave a knockdown of at least 70%, indicating that for some genes multiple highly effective sequences were obtained, e.g. PRKAR2A.

Comparison of ProLabel Assay With Western Blot Analysis

To confirm that loss of ProLabel activity is due to protein loss, knockdown of three proteins (STAT1, STAT6 and p38α/MAPK14) was reassessed by western blot analysis.

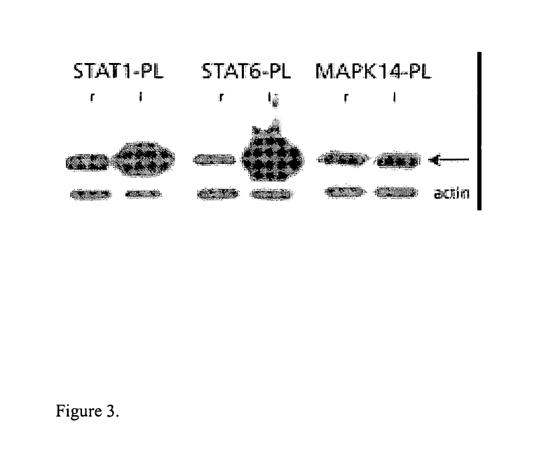

In this case, the respective ProLabel fusion construct for each of the proteins was co-transfected with either the irrelevant shRNA or the most effective of the 4 shRNAs originally tested. In all cases, knockdown observed by western blot was consistent with that determined using the ProLabel assay (compare FIGS. 2 and 3), being robust in the case of STAT1 and STAT6, and marginal for MAPK14. None of the experimental shRNAs had any observable effect by western blot on the level of endogenous β-actin expression, used as a loading control (FIG. 3). The slight apparent decrease in the expression of β-actin in the irrelevant shRNA control lane for STAT1 is presumably a loading artifact, since the same irrelevant shRNA control had no effect on the β-actin signal in the other two control lanes. In addition, no effect of the non-specific shRNA compared to mock transfected cells was seen (data not shown).

Comparison of ProLabel and Real-time Quantitative PCR Measurements

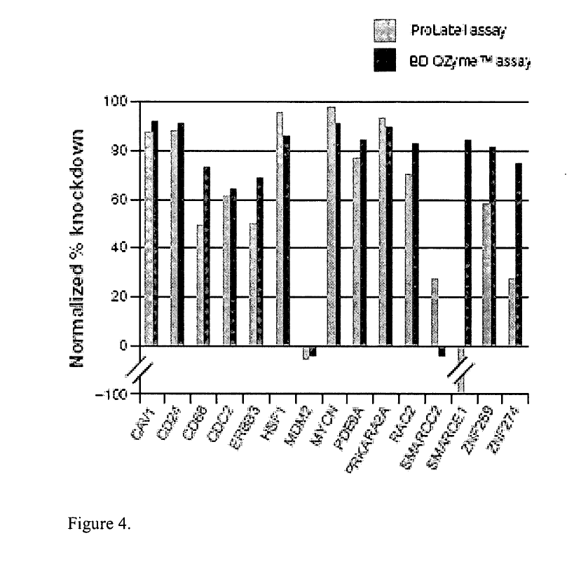

RNAi induced by siRNAs or shRNAs is believed to occur primarily through cleavage of the mRNA, preventing protein translation. To confirm that the ProLabel assay d at a reflected knockdown at the mRNA level, cell were co-transfected with a set of 15 ProLabel fusion vectors and corresponding shRNA expression vectors. After 48 hrs, the cells were collected, and a portion used to quantify mRNA expression levels by real-time RT-PCR. The remaining portion was used for ProLabel assays. In general, knockdown observed at the mRNA level, showed good correlation with the ProLabel data (FIG. 4). Protein knockdown levels were mostly lower than those obtained for mRNA, consistent with protein loss being secondary to mRNA cleavage. For 8 of the 15 genes tested (53%), the difference between the real-time PCR result and the ProLabel data was less than 15%. Another four genes (27%) showed less than 35% difference. Only three genes tested showed differences of more than 35%. All of these had either very low ProLabel expression (MDM2, SMARCE1) or low mRNA expression (SMARCC2, ZNF274), which may have confounded the accuracy of either measurement in these cases.

TABLE 1
Significance of various selection criteria on shRNA efficacy.

| | | Percentage of shRNAs with | | |
|---|---|---|---|---|
| | Calculated | given knockdown activity | | |
| Criterion analyzed | Probability | >50% | >70% | >90% |
| % GC > 52 | 0.007 | | | |
| shRNAs having this criterion | | 45% | 17% | 2% |
| shRNAs lacking this criterion | | 62% | 34% | 8% |
| 40 < GC % < 50 | 0.002 | | | |
| shRNAs having this criterion | | 64% | 35% | 8% |
| shRNAs lacking this criterion | | 48% | 20% | 4% |
| Presence of internal hairpins | 0.025 | | | |
| shRNAs having this criterion | | 51% | 20% | 4% |
| shRNAs lacking this criterion | | 62% | 34% | 7% |
| AA at positions 18, 19 | 0.045 | | | |
| shRNAs having this criterion | | 64% | 37% | 14% |
| shRNAs lacking this criterion | | 59% | 30% | 5% |
| G or C at positions 18, 19 | 0.063 | | | |
| shRNAs having this criterion | | 57% | 0% | 0% |
| shRNAs lacking this criterion | | 60% | 32% | 7% |
| A at position 3 | 0.289 | | | |
| shRNAs having this criterion | | 66% | 31% | 5% |
| shRNAs lacking this criterion | | 58% | 32% | 7% |
| U at position 10 | 0.212 | | | |
| shRNAs having this criterion | | 55% | 31% | 8% |
| shRNAs lacking this criterion | | 62% | 32% | 6% |
| A at position 6 | 0.018 | | | |
| shRNAs having this criterion | | 62% | 40% | 9% |
| shRNAs lacking this criterion | | 59% | 28% | 6% |
| G at position 13 | 0.046 | | | |
| shRNAs having this criterion | | 50% | 29% | 4% |
| shRNAs lacking this criterion | | 63% | 32% | 7% |
| All shRNAs | N/A | | | |
| shRNAs having this criterion | | 61% | 31% | 7% |

For all criteria analyzed, the probability that shRNAs having the given characteristic perform as effectively as those not having the characteristic was calculated using a Rank-Sum comparison. Also given in the table are the relative percentages of shRNAs with or without the given characteristic that fall within a given efficacy range.

It is evident from the above results that the subject method provides for a convenient way to screen RNAi molecules for activity. The method is simple, allows for rapid and accurate determinations, can be applied to high throughput screening and can use standard reagents and equipment for readout.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for screening RNAi molecules for inhibition of expression of a target protein, said method comprising:
introducing into a cell, which cell is transformed with a transcription construct for transcription of mRNA encoding a fusion protein comprising said target protein and a small fragment of β-galactosidase (“ED”), whereby expression of said fusion protein occurs, which small fragment is competent to complex with a large fragment of β-galactosidase to form an active enzyme;
introducing into said cell an RNAi molecule to determine the level of inhibition of said expression of said fusion protein by said RNAi molecule;
combining any of said fusion protein with EA and a β-galactosidase substrate producing a detectable product;
detecting the formation of said product;
whereby the level of product production is inversely related to the effectiveness of the RNAi inhibition of expression of said target protein.

2. A method according to claim 1, wherein said RNAi molecules are produced by a DNA construct transiently introduced into said cells.

3. A method according to claim 2, wherein said RNAi molecules are shRNA.

4. A method according to claim 1, wherein said RNAi molecules are siRNA.

5. A method according to claim 1, wherein said cells are mammalian cells.

6. A method for screening a plurality of RNAi molecules for inhibition of expression of a target protein, said method comprising:
introducing cells individually into a plurality of wells, said cells transformed with a transcription construct for transcription of mRNA encoding a fusion protein comprising said target protein and a small fragment of β-galactosidase (“ED”), whereby expression of said fusion protein occurs, which small fragment is competent to complex with a large fragment of β-galactosidase to form an active enzyme;
introducing into each of said cells in different wells different RNAi molecules to determine the level of inhibition of said expression of said fusion protein by said RNAi molecule for each of said RNAi molecules;
combining any of said fusion protein from each of said wells with EA and a β-galactosidase substrate producing a detectable product;
detecting the formation of said product from each of said wells;
whereby the level of product production in each of said wells is inversely related to the effectiveness of the RNAi inhibition of expression of said target protein.

7. A method according to claim 6, wherein said wells are microtiter plate wells.

8. A method according to claim 7, wherein robots are used for at least one stage to perform said method.

9. A method according to claim 6, wherein said RNAi molecules are produced by a DNA construct transiently introduced into said cells.

10. A method according to claim 9, wherein said RNAi molecules are shRNA.

11. A method according to claim 6, wherein said RNAi molecules are siRNA.

12. A method according to claim 6, wherein said cells are mammalian cells.


SEQUENCE LISTING (10 sequences)

SEQ ID NO: 1 - DNA, 20 bp, Artificial
  PCR Primer
actccgccccattgacgcaa

SEQ ID NO: 2 - DNA, 21 bp, Artificial
  PCR Primer
tccgctcatgagacaataacc

SEQ ID NO: 3 - DNA, 20 bp, Artificial
  PCR Primer
caccttgtcgccttgcgtat

SEQ ID NO: 4 - DNA, 20 bp, Artificial
  PCR primer
gacgattgacggcattacgt

SEQ ID NO: 5 - DNA, 20 bp, Artificial
  PCR primer
tgcctttgatgttcagcagg

SEQ ID NO: 6 - DNA, 8 bp, Artificial
  synthetic PCR primer
acacacac

SEQ ID NO: 7 - DNA, 8 bp, Artificial
  synthetic PCR primer
aaccaacc

SEQ ID NO: 8 - DNA, 24 bp, Artificial
  expression primer
cctgcgttatcccctgattctgtg

SEQ ID NO: 9 - DNA, 24 bp, Artificial
  primer
cagggcggggcgtaatttgatatc

SEQ ID NO: 10 - DNA, 19 bp, Artificial
  shRNA sense strand
gtgcgttgctagtaccaac